(19) United States
(12) Patent Application Publication
(10) Pub. No.: US 2015/0011653 A1
(43) Pub. Date: Jan. 8, 2015

(54) Novel Microbial Pathogen inhibitor

(71) Applicant: Abby Aronowitz, Huntington Station, NY (US)

(72) Inventor: Abby Aronowitz, Huntington Station, NY (US)

(73) Assignee: Abby Aronowitz, Huntington Station, NY (US)

(21) Appl. No.: 14/287,165

(22) Filed: May 26, 2014

Related U.S. Application Data
(60) Provisional application No. 61/842,607, filed on Jul. 3, 2013.

(51) Int. Cl. A61K 31/047; A61Q 19/00; A61K 8/34; A61K 9/00; A01N 31/02

(52) U.S. Cl. CPC A61K 31/047; A61K 9/0073; A61K 9/0031; A61K 9/0034; A01N 31/02; A61K 9/0053; A61K 9/0046; A61K 8/345; A61Q 19/00
USPC 514/738

(57) ABSTRACT

A formulation containing xylitol is used in a variety of ways to inhibit the ability of microorganisms to adhere to mucus membranes in the lungs throat, bladder, colon, ears, skin and rectum, and subsequently infect the host.

In this age of antibiotic resistant bacteria, xylitol is an inexpensive alternative, which does not create problematic resistant bacterial strains for E. coli, staph, strep and C diff.

### FIELD OF THE INVENTION

The field of the invention is anti-microbial therapy.

### BACKGROUND OF THE INVENTION

In U.S. Pat. No. 8,680,263, Kozlowski, et al. describe the use of compounds including polysaccharaides to form conjugates which can be used for drug delivery and purification. This is based upon the fact that polar organic functional groups can form weak interactions with drug molecules, which often have polar sections as well.

### OBJECTS OF THE INVENTION

One object of the invention is to develop a novel antimicrobial. Another object of the invention is to develop a preventative treatment for airborne pathogens to arrest epidemics. Yet another object of the invention is to develop a pathogen mitigating enema. Another object of the invention is to develop a pathogen mitigating solution to target bacteria in the ears, lungs, throat, colon, anus, and bladder. Another object of the invention is to develop delivery systems which would target specific, localized areas, such as an enema for the colon, peri-anal wipe for the anus, syringe for bladders, ear dropper for ears, inhaler or nebulizer for lungs, and spritz or pre-moistened towelette for cleaning surfaces. Another object is gargle for the throat, and a product combining zinc with xylitol, to minimize upper respiratory symptoms.

### SUMMARY OF THE INVENTION

The invention is the use of xylitol as a microbial infection inhibitor for non-sinus, non-oral, treatments.

### DETAILED DESCRIPTION OF THE INVENTION

Xylitol is a pentose polyol which has ligand-molecule interactions with many binding site proteins and polysaccharides found in pathogen attachment mechanisms. A solution of xylitol will therefore form complexes with the binding sites and inhibit microbial attachment. Attachment is the first step in many human pathogen infections by microbes.

### Examples of the Invention

In one preferred embodiment of the invention, a solution of 10% xylitol in water w/w is sprayed into the ambient air via a mister during an outbreak of airborne microbe borne infection epidemic.

In another preferred embodiment, the solution is injected into the lower colon via an enema to reduce colon mucus microbial attachment.

In yet another preferred embodiment the solution of xylitol is inhaled into the lungs via an atomizer to prevent or neutralize lung infections.

In yet another preferred embodiment, a xylitol paste with 95% xylitol w/w and 5% w/w alcohol is applied via application tube to the anus.

In yet another preferred embodiment, a 8% w/w xylitol in physiological buffer solution is introduced to the bladder via a catheter in a syringe to prevent bladder infection.

In yet another preferred embodiment, a solution of 10% w/w xylitol is blended with a variety of oils in an emulsion for use as a skin cream cosmetic with antimicrobial properties.

In yet another preferred embodiment, 10% w/w xylitol is mixed with a ethanol-water solution for use as hand sanitizer.

1. The use of xylitol as a microbial adhesion inhibitor for respiratory spray.

2. The use of xylitol as a microbial adhesion inhibitor for peri-anal pre-moistened wipes.

3. The use of xylitol as an inhaler delivered microbial inhibitor.

4. The use of xylitol as an enema solution for use in the colon.

5. The use of xylitol as a syringe delivered solution for intra-bladder use.

6. The use of xylitol as a wetting agent for towelettes to clean surfaces.

7. The use of xylitol as a microbial adhesion inhibitor for throat gargle use.

8. The use of xylitol for ear infection prevention in ear drops.

9. The claims in 1-8 where the solution ranges from 1% to 95% xylitol in water

10. The claims in 1-8 where the solution ranges from 1% to 95% xylitol in an alcohol.

11. The claims in 1-8 where the solution is a mixed solvent system.

12. The claims in 1-11 where zinc salts are added to the formulae.

13. The claims in 1-12 where the pathogen is gram negative bacteria.

14. The claims in 1-12 where the pathogen is a protozoan.

15. The claims in 1-12 where the pathogen is gram positive bacteria.

16. The claims in 1-12 where the pathogen is viral.

17. The claims in 1-10 where the solution is used as hand sanitizer.

18. The use of xylitol in a cosmetic lotion

19. Xylitol in claims 1-18 which blocks the attachment mechanisms for Streptococcus pneumonia, and Staphylococcus, Clostridium Difficile, and E. Coli.
